(19) United States
(12) Patent Application Publication
(10) Pub. No.: US 2009/0326321 A1
(43) Pub. Date: Dec. 31, 2009

(54) Miniaturized Imaging Device Including Multiple GRIN Lenses Optically Coupled to Multiple SSIDs

(76) Inventors: Stephen C. Jacobsen, Salt Lake City, UT (US); David L. Wells, Toronto (CA); David P. Marceau, Salt Lake City, UT (US)

Correspondence Address: THORPE NORTH & WESTERN, LLP., P.O. Box 1219, SANDY, UT, 84091-1219, US

(21) Appl. No.: 12/487,481

(22) Filed: Jun. 18, 2009

Related U.S. Application Data
(60) Provisional application No. 61/132,558, filed on Jun. 18, 2008.

(51) Int. Cl. A61B 1/05

(52) U.S. Cl. 600/109

(57) ABSTRACT

A miniaturized imaging device and method of viewing small luminal cavities are described. The imaging device can be used as part of a catheter, and can include at least one solid state imaging device (SSID) including multiple imaging arrays respectively, and multiple graduated refractive index (GRIN) lenses optically coupled to the multiple imaging arrays.

### PRIORITY

This application claims the benefit of U.S. Provisional Application No. 61/132,558 filed on Jun. 18, 2008 entitled “Miniaturized Imaging Device Including Multiple GRIN Lenses Optically Coupled to Multiple SSIDs” the entirety of which is incorporated herein by reference.

### FIELD OF THE INVENTION

The present invention relates to medical devices, and more particularly to miniaturized in-situ imaging devices and methods of operation of said devices.

### BACKGROUND OF THE INVENTION

The invention relates generally to solid state imaging devices (SSIDs). More specifically, the invention relates to miniaturized imaging devices that are particularly suited to viewing beyond small openings and traversing small-diameter areas. These devices can be used for catheter-borne medical imaging within the anatomy of a patient, and are useful for other applications.

Small imaging devices that take advantage of advances in integrated circuit imaging technologies are known. Such small imaging devices can be particularly useful in medical diagnostic and treatment applications. Portions of human anatomy previously viewable only by a surgical procedure can be viewed now by a minimally invasive catheterization, provided an imaging device can be made that is small enough to view the target anatomy.

Other uses for very small imaging devices are recognized. For example, such devices can be used and are desirable for surveillance applications, for monitoring of conditions and functions within devices, and for size- and weight-critical imaging needs as are present in aerospace applications, to name a few.

While the present invention has applications in these aforementioned fields and others, the medical imaging application can be used to favorably illustrate unique advantages of the invention. The desirability of providing imaging at sites within the anatomy of living creatures, especially humans, distal of a small orifice or luminal space has long been recognized. A wide variety of types and sub-types of endoscopes have been developed for this purpose.

One advance in imaging technology which has been significant is in the area of SSIDs. Such devices, including the charge-injection device (CID), the charge-coupled device (CCD), and the complementary metal oxide semiconductor (CMOS) device, provide good alternatives to the use of bundled fiber optics, as well as to conventional miniaturized imaging devices used in endoscope applications. However, when considering a design of a catheter-borne imaging device, consideration should be given to the ability of a distal tip of the catheter to flex and bend, without breaking or becoming damaged. This is necessary to accommodate limitations of anatomy to minimize trauma, and to enable steering of the distal tip to a desired location.

### SUMMARY OF THE INVENTION

It has been recognized that by looking outside conventional devices and techniques, that facilitation of further miniaturization of an imaging device employing SSIDs at a distal end of a catheter or other flexible umbilical can be accomplished. The invention accordingly provides a miniaturized imaging device, comprising at least one SSID including multiple imaging arrays, and multiple GRIN lenses optically coupled to the multiple imaging arrays of the at least one SSID, respectively. A GRIN lens is defined as a graduated refractive index lens.

In accordance with the invention as embodied and broadly described herein, the present invention resides in a miniature imaging device comprising a catheter having a distal end and a proximal end, a first imaging system disposed on the distal end of the catheter, the first imaging system having a level of magnification and a field of view and comprising an imaging array disposed on an SSID and a GRIN lens disposed on a surface of the imaging array. The invention further comprises a second imaging system disposed on the distal end of the catheter and parallel to the first imaging system, the second imaging system having a level of magnification and field of view and comprising an imaging array disposed on an SSID and a GRIN lens disposed on a surface of the imaging array. Further, the level of magnification of the first imaging system is greater than the level of magnification of the second imaging system and the field of view of the first imaging system is less than the field of view of the second imaging system.

In accordance with another embodiment of the present invention, a miniature imaging device comprises a miniature capsule body and a plurality of imaging arrays disposed on a plurality of SSIDs respectively. The plurality of imaging arrays are positioned about the miniature capsule body to provide a plurality of non-parallel views. The invention further comprises a plurality of GRIN lenses optically disposed in direct contact with a top surface of the plurality of imaging arrays and configured such that a distal end of each of the GRIN lenses is substantially coplanar with an outer surface of the capsule body.

In accordance with another embodiment of the present invention, a miniature imaging device comprises a miniature capsule body and an SSID having a plurality of non-parallel sides, said SSID enclosed within the miniature capsule body. The invention further comprises an imaging array disposed on each of the non-parallel sides of the SSID and a single GRIN lens optically coupled to each of the imaging arrays and oriented substantially within the capsule body.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention they are, therefore, not to be considered limiting of its scope. It will be readily appreciated that the components of the present invention, as generally described and illustrated in the figures herein, could be arranged and designed in a wide variety of different configurations. Nonetheless, the invention will be described and explained with additional specificity and detail through the use of the accompanying drawings in which:

FIG. 1 is a schematic illustration of an exemplary medical imaging system in accordance with principles of the invention;

FIG. 2 is a side view of an exemplary embodiment of the present invention, which is an enlarged view of device 14 of FIG. 1;

FIG. 3 is a perspective view of another exemplary embodiment of the invention;

FIG. 4 is a top view of the device of FIG. 3;

FIG. 5 is a side view of the device of FIG. 3, rotated 90 degrees with respect to FIG. 4;

FIG. 6 is a cross sectional view of another exemplary embodiment of the invention;

FIG. 7 is a cross sectional view of another exemplary embodiment of the invention;

FIG. 8 is a cross sectional view of another exemplary embodiment of the invention in a first configuration;

FIG. 9 is a cross-sectional view of the device of FIG. 8 in a second position view;

FIG. 10 is a perspective view of an SSID optically coupled to a GRIN lens;

FIG. 11 is a perspective view of an exemplary embodiment of an SSID and multiple GRIN lenses positioned in an array;

FIG. 12 is a perspective view of another exemplary embodiment of an SSID and multiple GRIN lenses positioned in an array;

FIG. 13 is a side view of multiple microcameras positioned along an umbilical as an array;

FIG. 14 is plan view along the optical axis of an exemplary color filter insert that can be used with imagine devices in accordance with principles of the invention;

FIG. 15 is a first side view of the color filter insert of FIG. 14;

FIG. 16 is a second side view of the color filter insert of FIG. 14, taken at 90 degrees with respect to FIG. 15;

FIG. 17 is a schematic side view representation of another exemplary embodiment having a color filter insert of FIG. 14 inserted therein;

FIG. 18 is a schematic side view representation of another exemplary embodiment having a fiber optic inserted therein;

FIG. 19 is a perspective view of an exemplary embodiment of multiple SSIDs and multiple GRIN lenses positioned in an array;

FIG. 20 is a perspective view of another exemplary embodiment of multiple SSIDs and multiple GRIN lenses positioned in an array;

FIG. 21 is a side view of another exemplary embodiment of multiple microcameras positioned along an umbilical as an array;

FIG. 22 is a side view of another exemplary embodiment of multiple microcameras positioned circumferentially around an umbilical as an array;

FIG. 23 is a perspective view of an exemplary embodiment of a mini-capsule having multiple SSIDs and multiple GRIN lenses positioned in an array; and

FIG. 24 is a perspective view of an exemplary embodiment of a mini-capsule having multiple SSIDs and multiple GRIN lenses positioned in an array.

### DETAILED DESCRIPTION OF THE PREFERRED EMBODIMENT(S)

The following detailed description of exemplary embodiments of the invention makes reference to the accompanying drawings, which form a part hereof and in which are shown, by way of illustration, exemplary embodiments in which the invention may be practiced. While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

The following detailed description and exemplary embodiments of the invention will be best understood by reference to the accompanying drawings, wherein the elements and features of the invention are designated by numerals throughout.

It must be noted that, as used in this specification and the appended claims, singular forms of “a,” “an,” and “the” include plural referents unless the context clearly dictates otherwise.

An “SSID,” “solid state imaging device,” or “SSID chip” in the exemplary embodiments generally comprises an imaging array or pixel array for gathering image data, and can further comprise conductive pads electrically coupled to the imaging array, which facilitates electrical communication therebetween. In one embodiment, the SSID can comprise a silicon chip substrate or other semiconductor chip substrate (e.g., InGaAs) or amorphous silicon thin film transistors (TFT) having features typically manufactured therein. The SSID can also comprise a non-semiconductor chip substrate treated with a semiconductor material. Features can include the imaging array, the conductive pads, metal traces, circuitry, etc. Other integrated circuit components can also be present for desired applications. However, it is not required that all of these components be present, as long as there is a means of gathering visual or photon data, and a means of sending that data to provide a visual image or image reconstruction.

The term “umbilical” can include the collection of utilities that operate the SSID or the micro-camera as a whole. Typically, an umbilical includes a conductive line, such as electrical wire(s) or other conductors, for providing power, ground, clock signal, and output signal with respect to the SSID, though not all of these are strictly required. For example, ground can be provide by another means than through an electrical wire, e.g., to a camera housing such as micromachined tubing, etc. The umbilical can also include other utilities such as a light source, temperature sensors, force sensors, fluid irrigation or aspiration members, pressure sensors, fiber optics, microforceps, material retrieval tools, drug delivery devices, and radiation emitting devices, laser diodes, electric cauterizers, and electric stimulators, for example. Other utilities will also be apparent to those skilled in the art and are thus comprehended by this disclosure. Despite specific reference to a light source carried by the utility, it is understood that light sufficient to image a target could be generated through fluorescence or other source as understood in the art.

“GRIN lens” or “graduated refractive index lens” refers to a specialized lens that has a refractive index that is varied radially from a center optical axis to the outer diameter of the lens. In one embodiment, such a lens can be configured in a cylindrical shape, with the optical axis extending from a first flat end to a second flat. Thus, because of the differing refractive index in a radial direction from the optical axis, a lens of this shape can simulate the affects of a more traditionally shaped lens.

With these definitions in mind, reference will now be made to the accompanying drawings, which illustrate, by way of example, embodiments of the invention.

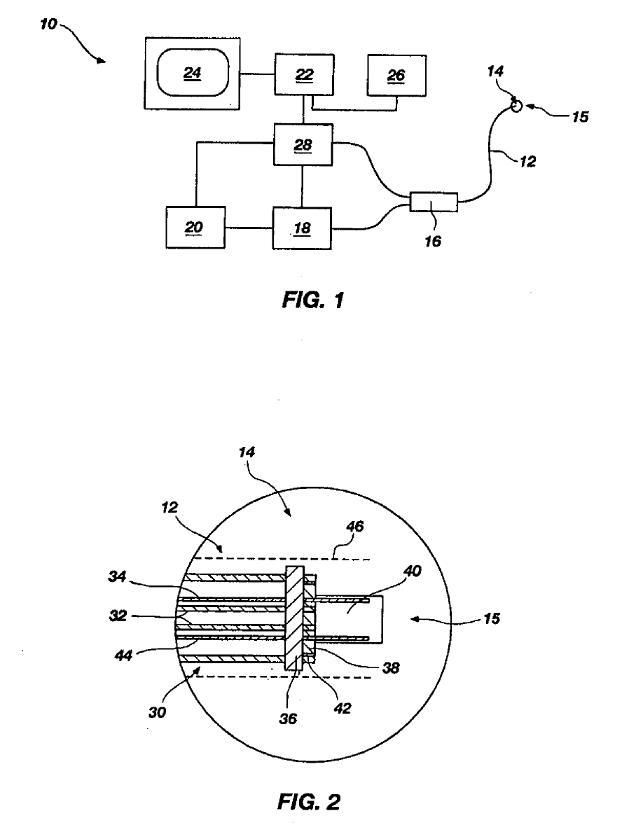

With reference to FIGS. 1 and 2, the invention is embodied in a medical imaging system 10, including a catheter 12 having an imaging capability by means of an imaging device, shown generally at 14, at a distal tip 15 of the catheter. The system further includes a fitting 16 enabling an imaging fluid, such as a clear saline solution, to be dispensed to the distal tip portion of the catheter from a reservoir 18 to displace body fluids as needed to provide a clearer image. A pump 20 is provided, and is manually actuated by a medical practitioner performing a medical imaging procedure, or can be automated and electronically controlled so as to dispense fluid on demand according to control signals from the practitioner, sensors, or according to software commands.

A processor 22, such as an appropriately programmed computer, is provided to control the imaging system 10 and create an image of anatomy adjacent the distal tip portion 15, within a patient (not shown), displayable on a monitor 24, and storable in a data storage device 26. An interface 28 is provided which supplies power to the imaging device 14 and feeds a digital image signal to the processor based on a signal received from the imaging device via an electrical umbilical 30, including conductive wires 32, a fluid dispenser 34, and a light source 44, through the catheter 12. The interface can also be configured to control the pump 20 based on control signals from the processor or a medical practitioner performing an imaging procedure.

With more specific reference to FIG. 2, the imaging device 14 at the distal tip 15 can include a utility guide 36 for supporting or carrying the umbilical 30, which can include electrical wires 32, a fluid dispenser 34, and a light source 44. Other components that can be carried by the utility guide can include, temperature sensors, force sensors, fluid irrigation or aspiration members, pressure sensors, fiber optics, microforceps, material retrieval tools, drug delivery devices, radiation emitting devices, laser diodes, electric cauterizers, and electric stimulators. The utility guide can also carry an SSID or solid state imaging device 38 that includes an imaging array (not shown) and conductive pads 42 for coupling the electrical wires to the SSID. The light source shown is a fiber optic carried by the utility guide. However, other light sources can be used, such as those carried by the SSID. For example, the SSID can also include light-emitting diodes (LEDs) configured to illuminate the area immediately adjacent the distal tip portion. With the SSID in this configuration, a GRIN rod lens 40 is shown optically coupled to the imaging array of the SSID.

The GRIN rod lens 40 can be substantially cylindrical in shape. In one embodiment, the GRIN rod lens can have a first flat end for receiving light, a second flat end for passing the light to the imaging array, and an outer curved surface surrounded by an opaque coating or sleeve member to prevent unwanted light from entering the GRIN rod lens. The GRIN rod lens can be optically coupled to the imaging array by direct contact between the second flat end and the imaging array of the SSID 38. Such direct contact can include an optically transparent or translucent bonding material at the interface between the second flat end and the imaging array. Alternatively, the GRIN rod lens can be optically coupled to the imaging array of the SSID through an intermediate optical device, such as a fiber optic or a color filter, or any shape optical lens such as a prism or wide angle lens.

The catheter 12 can be configured to be bendable and flexible so as to be steerable within a patient's anatomy and to minimize trauma. For example, the catheter can comprise a micromachined tube 46 at the distal tip portion, and cut-out portions (not shown) can allow for increased flexibility of the tube, and also allow for outflow of an imaging fluid to displace body fluids in the immediate area of the distal tip portion for clearer imaging. Such a micromachined tube can also allow bending to facilitate guiding the catheter to a desired location by selection of desired pathways as the catheter is advanced. Additional details on construction of similar slotted micro-machined tube or segments can be found in U.S. Pat. Nos. 6,428,489, which is incorporated herein by reference.

The catheter 12 can alternatively comprise an internal tensionable wire (not shown) adjacent one side of the distal tip portion, which when tensioned, causes the distal tip portion 15 to deflect as is known in the art. A combination of deflection and rotation of the distal tip portion of the catheter provides steer-ability of the device. Another alternative for directability of the distal tip portion is to provide a micro-actuator (not shown) such as an element which expands or contracts upon application of an electrical current signal. Such an element can be substituted for the tension wire, for example.

As will also be appreciated, while the system is illustrated by the exemplary embodiment of a medical imaging system, these arrangements could be used in other devices, such as visual sensors in other devices, surveillance apparatus, and in other applications where a very small imaging device can be useful.

Moreover, with reference to all of the embodiments described herein, the device contemplated can be very small in size, and accordingly the imaging array of the SSID can have a lower pixel count than would otherwise be desirable. As technology advances, pixel size can be reduced, thereby providing clearer images and data. However, when using a lower number of pixels in an imaging array, the resolution of the image provided by the device can be enhanced through software in processing image data received from the SSID. The processor showing in FIG. 1, can be appropriately programmed to further resolve a scanned image from an array of an SSID, for example, based on information received as the SSID is moved slightly, such as from vibration controlled vibration. The processor can analyze how such image data from the imaging array is altered due to the vibration, and can refine the image based on this information.

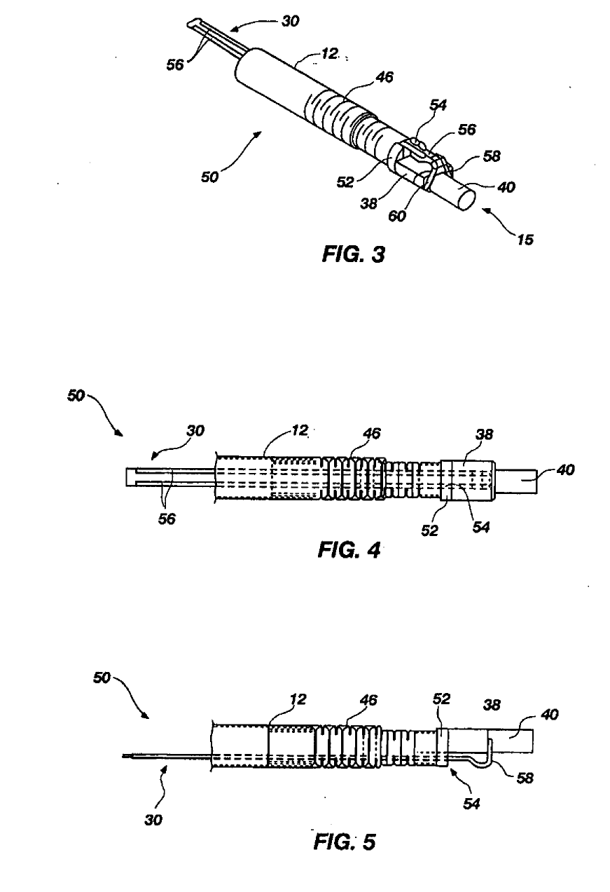

Turning now to FIGS. 3 to 5, another embodiment of the invention is implemented as shown in system 50, wherein a distal tip portion 15 of a catheter 12 includes lens 40 optically coupled to an SSID 38. Here, the SSID is also electrically bonded to an adaptor 52. The adaptor is carried by micromachined tubing segment 46, and is configured to fit within it at a distal end of the tubing segment. The adaptor has a channel 54 formed therein which allows passage of a conductive strip 56 (which functions similarly as the conductive wires of FIG. 2) of an umbilical 30. The micromachined tubing segment itself is configured to provide telescoping action. This allows the distal tip portion of the catheter to be assembled and then connected easily to the remainder of the catheter. The conductive strip can comprise a ribbon formed of a non-conductive material, for instance a polyimide film such as KAPTON™, with conductive traces overlain with a dielectric, and provides an electrical umbilical to the SSID through the adaptor. The conductive strip can be threaded back through the catheter to a fitting (not shown) at its proximal end, as discussed previously. At a distal portion of the conductor strip, individual conductor elements 58, 60 are separated from the non-conductive strip and are bonded to conductive pads (not shown in FIG. 3-5) that are present on the adaptor. Thus, the adaptor provides a power conduit from the umbilical to the SSID. Additional principles of operation and details of construction of similar micro-camera assemblies can be found in U.S. patent application Ser. Nos. 10/391,489, 10/391,490, 11/292,902, and 10/391,513 each of which are incorporated herein by reference in their entireties.

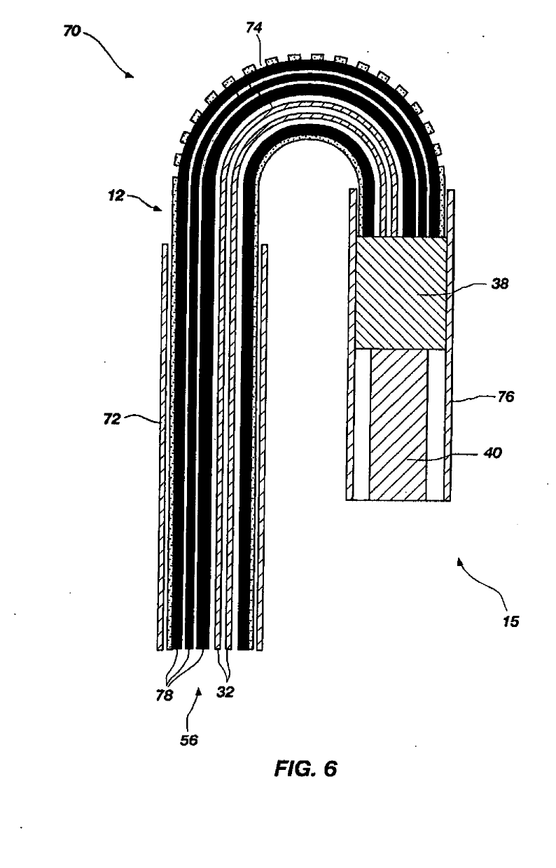

With reference to FIG. 6, another system is shown generally at 70. In this embodiment, the distal tip 15 of the catheter 12 is shown. An outer sleeve 72 is provided over the outside of the catheter in telescoping fashion. The catheter can be withdrawn into the sleeve at will by differential movement at a proximal end (not shown) of the device. An outer tubing of the catheter can be micromachined to provide a pre-disposition to bend adjacent the SSID 38, for example by micomachining the tubing to provide openings 74 on one side of the tubing and bending the tubing to give it a curved configuration doubling back on itself as shown in the figure. The tip can be directed as desired by pulling the curved portion of the catheter partially, or completely, back into the outer sleeve. In one embodiment, the micro-machined tubing is formed of super-elastic material with embedded shape memory capability, such as NiTi alloy so that this can be done repeatedly without the material taking a set. A further outer sleeve 76 is provided adjacent the SSID and GRIN rod lens 40 to support this structure. A conductive strip 56, including conductive wires 32, can be provided, as described previously.

In another embodiment, tensioning wires 78 can be provided in a lumen within the catheter adjacent a large radius, or outer portion of the catheter 12, which enables directing the tip 15 by providing a tension force tending to straighten out this portion of the catheter. The tension wire is attached to the SSID 38 and extends back through the catheter to a proximal portion where it can be manipulated by a practitioner doing the imaging procedure. The catheter can also include provision for supplying imaging fluid, light, or other utilities, as discussed above.

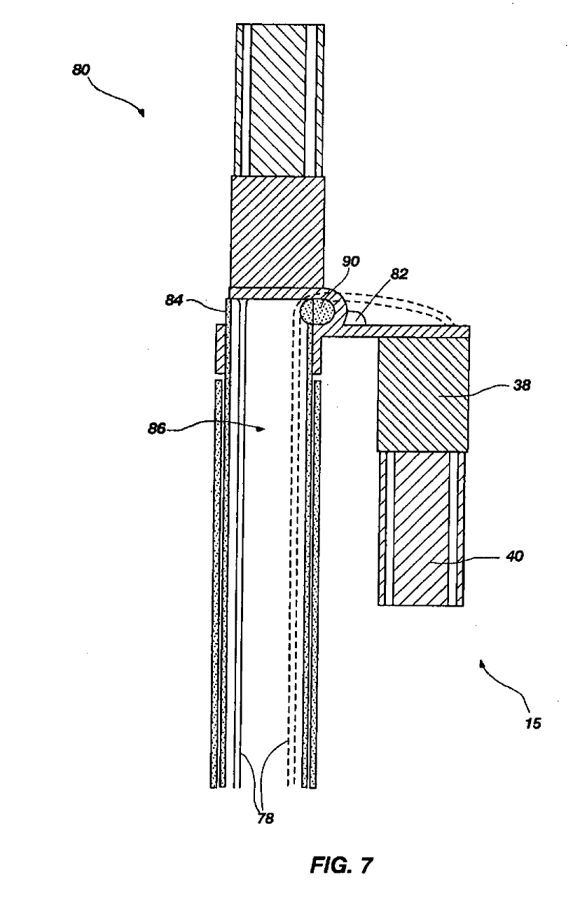

With reference to FIG. 7, a system shown generally at 80, can comprise an SSID 38 mounted on a hinge 82 formed of super-elastic material with embedded shape memory capability. The hinge is connected to a tube 84 defining an inner lumen 86 of the catheter 12. Tensioning wires 78 are attached to the hinge, and allows the SSID to be directed from a first direction aimed back along the longitudinal axis of the catheter, through 180 degrees, to a second position aiming distally away from the catheter in a direction substantially coincident with the longitudinal axis. This, in combination with rotation of the catheter, allows for directability of the tip. A rounded guide 90 is attached to a distal portion of the tube to provide a radius for the tensioning wires and the hinge so that they do not kink, but deform elastically as shown. Conductive wires (not shown) can be present as describe previously.

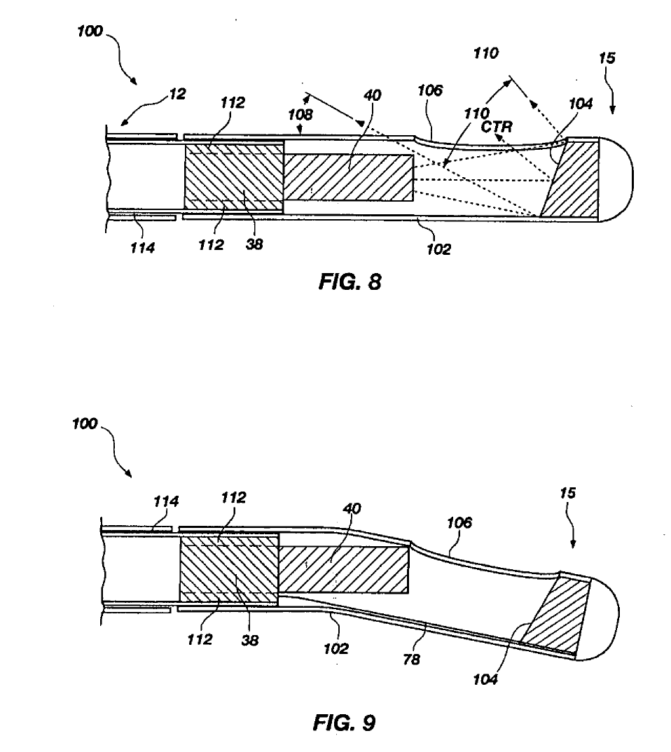

Continuing now with reference to FIGS. 8 and 9, an alternative system is shown generally at 100. As shown, control means for directing the catheter 12 and/or directing the field of view of the SSID 38 at the distal tip portion 15 of the catheter is illustrated. A deformable outer sleeve 102 comprising a mirror element 104 at a distal end is provided. An opening 106 adjacent the mirror element and the GRIN rod lens 40 enables appropriate imaging.

In one configuration state, shown in FIG. 8, the angled surface of the mirror allows a view rearwardly and to the side of the catheter at an angle 108 of about 25 to 50 degrees with respect to a longitudinal axis of the catheter. A field of view 110 based on the configuration and spacing, and angular relationships between the elements can comprise between about 15 and 25 degrees. The SSID can comprise one or more lumens 112 for conveying imaging fluid to the distal tip portion of the catheter, or to carry power to the imaging array (not shown) of the SSID. As will be appreciated, imaging fluid could also be conveyed to the imaging site via another lumen 114 or a guiding catheter, or a completely separate catheter (not shown).

In another configuration state, shown in FIG. 9, the deformable outer sleeve 102 is bent, enabling direct viewing forwardly through the opening 106. Also, views rearwardly at various angles can be obtained by causing more or less deflection of the deformable outer sleeve 102. Attached to the tube adjacent one side (a bottom side in FIG. 9), a tension wire 78 deflects the deformable outer sleeve as tension is applied. Another way for deforming the sleeve is to form it from a NiTi alloy, which changes shape from a first configuration shown in FIG. 8 to a second configuration in FIG. 9 via change of temperature such as can be affected by introduction of imaging fluid of a different temperature, or by running an electrical current therethrough. In the latter two embodiments, the tip has essentially two states, deformed and undeformed.

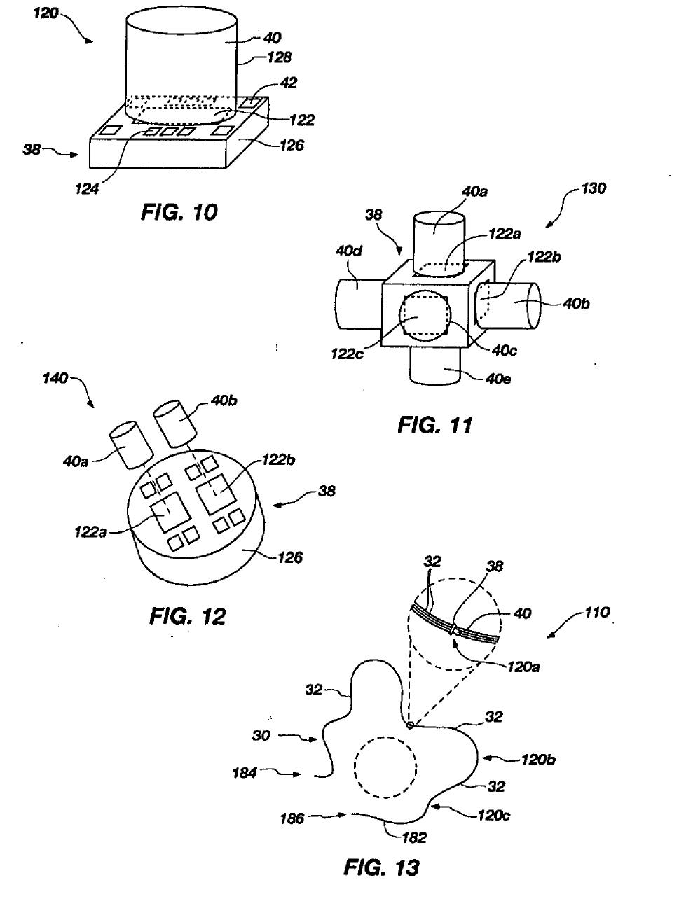

Referring now to FIG. 10, a system, indicated generally at 120, includes a GRIN lens 40 and an SSID 38. The SSID can comprise a silicon or other semiconductor substrate or amorphous silicon thin film transistors (TFT) 126 having features typically manufactured therein. The SSID can also comprise a non-semiconductor substrate coated with a semiconductor material or any other equivalent structure. Features including the imaging array 122, the conductive pads 42, metal traces (not shown), circuitry (not shown), etc., can be fabricated therein. With respect to the conductive pads, the connection between conductive pads and a conductive line of an umbilical (not shown) can be through soldering, wire bonding, solder bumping, eutectic bonding, electroplating, and conductive epoxy. However, a direct solder joint having no wire bonding between the electrical umbilical and the conductive pads can be preferred as providing good steer-ability can be achieved with less risk of breaking electrical bonding. In one embodiment, the conductive line of the umbilical can provide power, ground, clock signal, and output signal with respect to the SSID. Other integrated circuit components can also be present for desired applications, such as light emitting diodes (LEDs) 124, for providing light to areas around the GRIN rod lens.

It is not required that all of these components be present, as long as there is a visual data gathering and sending image device present, and some means provided to connect the data gathering and sending device to a visual data signal processor. Other components, such as the umbilical, housing, adaptors, utility guides, and the like, can also be present, though they are not shown in FIG. 10. The SSID 38 can be any solid state imaging device, such as a CCD, a CID, or a CMOS imaging device. Also shown, the GRIN rod lens 40 is coated with an opaque coating 128 on the curved surface to prevent light from entering the lens at other than the flat surface that is most distal with respect to the SSID.

FIG. 11 depicts an alternative system 130 that includes multiple imaging arrays 122a, 122b, 122c on a common SSID 38. Though only three imaging arrays are shown in this perspective view, five imaging arrays are present in this embodiment (i.e., one on each side of five sides the substrate 126, with the back side of the substrate providing a surface for umbilical connection). Each imaging array is respectively optically coupled to a GRIN lens 40a, 40b, 40c, 40d, 40e. As can be appreciated, this is but one configuration where multiple imaging arrays with multiple GRIN lenses can be used. Fewer or more imaging arrays can be used in other similar embodiments, and/or can be part of multiple SSIDs, as will be described in greater detail below. Umbilical connections are not shown, though it is understood that an umbilical can be present to operate the SSID and its multiple imaging arrays (either by signal splitting or by the use of separate power and/or signal sources). Additionally, a wireless transmitter can be included with this and other exemplary systems described, for transmitting image information to a remote receiver. A wireless transmitter can be included as a substitute for the umbilical or as an additional component.

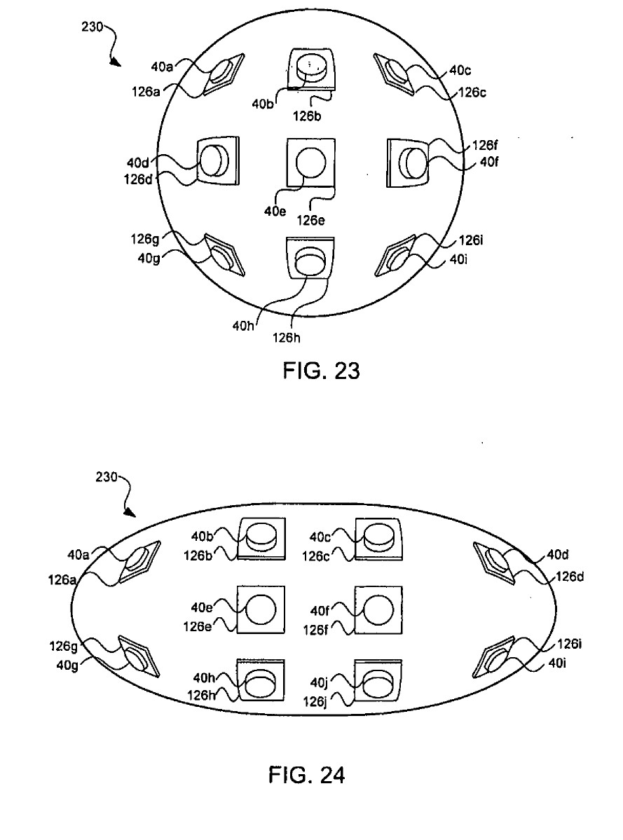

FIG. 23 depicts a substantially spherical capsule-type imaging system 230, shown generally at 230, for multi-directional imaging. A capsule body can be configure to carry multiple SSIDs 126a, 126b, 126c, 126d, 126e, 126f, 126g, 126h, 126i each having multiple imaging arrays (not shown). Multiple GRIN rod lenses 40a, 40b, 40c, 40d, 40e, 40f, 40g, 40h, 40i are shown as they would be optically coupled to the imaging arrays that are carried by the SSIDs. While capsule endoscopes are known in the art, these devices typically include pill-sized devices that only capture one to two angled views from the capsule. Thus, by including multiple SSIDs each laving an imaging array and GRIN rod lens, multiple fields of view can be acquired by the device, while maintaining a substantially small size. Additionally, the miniature imaging device can include a wireless transmitter for sending signals from the imaging array to a remote receiver as will be apparent to one of ordinary skill in the art. As noted below and as shown in FIG. 22, in one embodiment, the SSID/GRIN lens combination (e.g., the 126a/40a combination) is configured within the capsule such that a distal surface of the GRIN lens 40a is flush (i.e., coplanar) with an outer surface of the capsule.

In one aspect of the invention, the GRIN lens 40a is disposed within apertures in an outer surface of the capsule. However, in another aspect of the invention, a distal surface of the GRIN lens 40a is not flush with an outer surface of the capsule but is positioned directly behind a transparent section disposed about an outer surface of the capsule. Advantageously, the capsule may be constructed such that its outer surface has no apertures and is therefore less subject to fluid intrusion or contamination while inside the body.

With reference to FIG. 11 and FIG. 23, in one embodiment of the present invention a single SSID 38 having multiple imaging arrays oriented in a non-parallel orientation 122a, 122b may be disposed within a spherical capsule as shown in FIG. 23. While a cube structure is specifically shown in FIG. 11, it is understood and contemplated herein that numerous shapes and/or platonic structures could be used with embodiments of the invention without deviating from the principle of operation (e.g., a tetrahedron, octahedron, or dodecahedron).

In one embodiment, a plurality of capsules 230 could be consumed by a patient at the same time or at timed intervals. As the pills travel through the gastrointestinal system of the patient, the capsules 230 are configured to transmit wireless signals to each of the plurality of capsules within the gastrointestinal system as well as a receiver disposed outside of the patient in a fixed location. In this manner, the location of each of the plurality of capsules within the patient may be tracked relative to the receiver and relative to each other. Accordingly, images received from each of the plurality of capsules 230 may be more accurately correlated to specific locations within the patient.

FIG. 24 depicts a capsule-type endoscope system 240 having a common pill-shaped shape, similar in function to that depicted in FIG. 23 including multiple SSIDs 126a, 126b, 126c, 126d, 126e, 126f, 126g, 126h, 126i, 126j each having multiple imaging arrays (not shown) and multiple GRIN rod lenses 40a, 40b, 40c, 40d, 40e, 40f, 40g, 40h, 40i, 40j. It will be appreciated that a variety of other capsule shapes and sizes are well known as pill shapes in the pharmaceutical industry. Additionally, various shapes and sized of SSID devices can manufactured as is known in the art. The capsule may also comprise LED's or other internal light sources as described more fully herein. A similar configuration as noted above with respect to the single SSID structure shown in FIG. 11 may be utilized in connection with the pill-shaped device of FIG. 24.

Alternatively, the capsule-type imaging systems 230, 240 can include an umbilical for powering and receiving signal from imaging array through the conductive pads.

FIG. 12 depicts a system, shown generally at 140, which can provide stereoscopic imaging. Specifically, multiple imaging arrays 122a, 122b, are shown on a common SSID 38 in a coplanar arrangement. A pair of GRIN rod lenses 40a, 40b are shown as they would be optically coupled to imaging arrays 122a, 122b, respectively. Other than the imaging array, other features are also present in the SSID, including conductive pads 42 for providing an electrical connection to an umbilical (not shown).

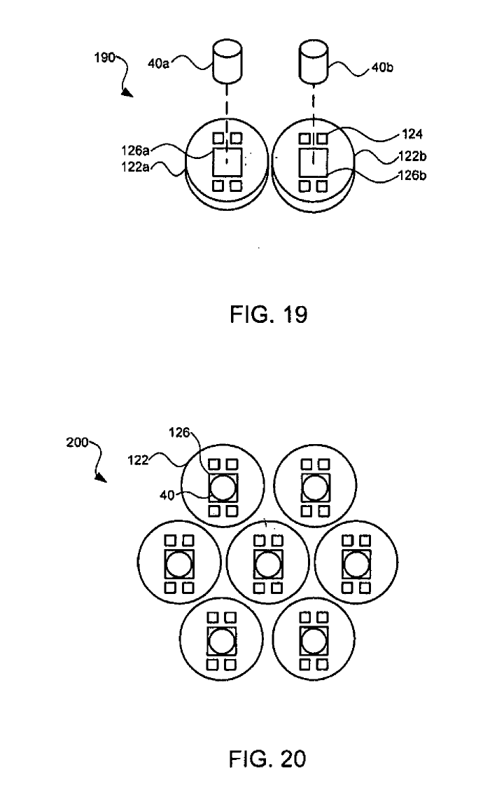

FIG. 19 depicts an alternate system, shown generally at 190, for providing stereoscopic imaging. Specifically, two imaging arrays 122a, 122b, are shown on two SSIDs 38a, 38b respectively, in a coplanar arrangement. A pair of GRIN rod lenses 40a, 40b are shown as they would be optically coupled to imaging arrays 122a, 122b, respectively. The coplanar, stereoscopic embodiments can improve the depth perception of the miniature imaging device as well as provide for higher definition resolution. Because of their small size, the SSID pair can be included on the distal end of a single catheter umbilical or utility guide, for example they can be coupled to the utility guide.

Turning now to FIG. 20, a system, shown generally at 150, can provide multi-camera imaging. Specifically, multiple imaging arrays 122a, 122b, 122c, 122d, 122e are shown on multiple SSIDs 38a, 38b, 38c, 38d, 38e in a coplanar arrangement. Multiple GRIN rod lenses 40a, 40b, 40c, 40d, 40e are shown as they would be optically coupled to imaging arrays 122a, 122b, 122c, 122d, 122e, respectively. This arrangement can provide improved resolution over that of the stereoscopic arrangement of FIGS. 12 and 19 as well and enhanced depth perception for the miniature imaging system.

Referring now to FIG. 13, a system 110 includes multiple microcameras 120a, 120b, 120c positioned along an umbilical 30, which are attached to conductive wires 32 of the umbilical. The umbilical includes a proximal end 184, which can be coupled to a processor/monitor (not shown) for viewing, and a distal end 186. Each microcamera includes an SSID 38 and a GRIN rod lens 40. In the embodiment shown, the microcamera 120c that is closest to a terminal end 186 is optically coupled to a fiber optic line 182, which can include a GRIN lens at a terminal end of the fiber optic line, as shown in FIG. 18 below. However, the microcamera closest to the terminal end can actually be at a distal tip of the catheter. To illustrate an approximation of the size of the microcameras of the present invention, structure 188 is shown, which is approximately the size of a small coin, such as a United States dime.

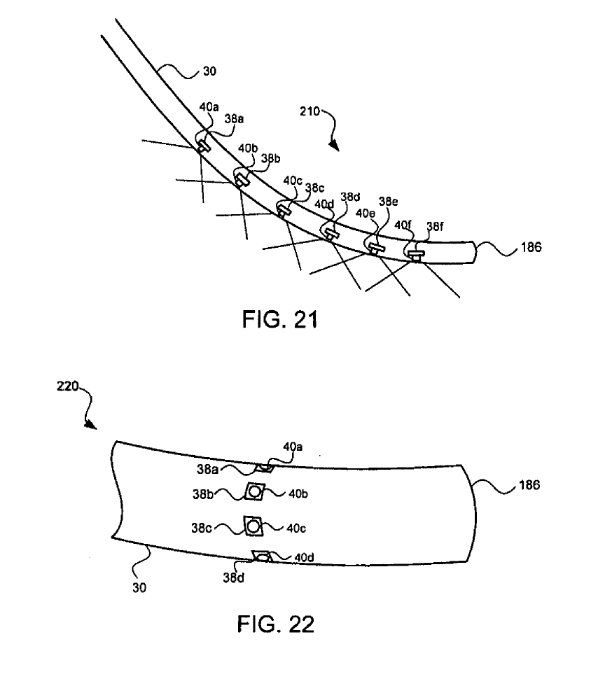

Referring now to FIG. 21, a system 210 includes multiple microcameras 120a, 120b, 120c, 120d, 120e, 120f positioned along an umbilical 30, similar to those of FIG. 13. Each microcamera includes an SSID 38 and a GRIN rod lens 40. In the embodiment shown, the microcameras are positioned so as to continuously image a lateral portion of the surrounding environment and/or tissue. Because catheter and endoscope procedures frequently are used to image the physical condition of internal passageways, for example the walls of veins and arteries, it can be advantageous to be able to obtain a continual lateral image without the necessity of physically turn a microcamera that is disposed on the distal end of the catheter 186. In addition, being able to obtain a continuous image of a passage can greatly enhance a physician's ability to recognize gradual changes and repeated problems in an internal passage. With specific reference to FIG. 21, in one embodiment, the SSID/GRIN lens arrangement (e.g., the 38a/40a combination) is configured such that the distal surface of the GRIN lens 40a is flush (i.e., coplanar) with an outer surface of the catheter 30. That is, the GRIN lens 40a is disposed within an aperture of the outer surface of the catheter 30. In an additional aspect, the GRIN lens 40a is disposed such that a distal end of the GRIN lens 40a corresponds with a transparent window disposed within an outer surface of the catheter 30.

Referring now to FIG. 22, a system 220 includes multiple microcameras 120a, 120b, 120c, 120d positioned circumferentially around an umbilical 30. Each microcamera includes an SSID 38 and a GRIN rod lens 40. This embodiment provides an additional benefit to the miniature imaging system. By being able to image an entire vein segment a physician can obtain multiple focused images of potentially problematic tissue. Such lateral images can image lesions, plaque, and other damaged of diseased tissue directly, as opposed to the forward view provided by an imaging device on the front end of a catheter or endoscope. In the figure a single ring of microcameras is shown, however, multiple rings of cameras can be positioned along the umbilical to image multiple areas of an internal passage.

The embodiments thus far shown depict GRIN rod lenses optically coupled to imaging arrays of SSIDs by a direct bonding or coupling. However, the term “optically coupled,” also provides additional means of collecting light from GRIN rod lens and coupling it to an imaging array of an SSID. For example, other optical devices can be interposed between a GRIN rod lens and an SSID, such as a color filter, fiber optic, or any shape optical lens including a prism or wide angle lens. Specifically, a system of converting monochrome imaging to multiple colors can be accomplished by utilizing a filter having a predetermined pattern, such as a Bayer filter pattern. The basic building block of a Bayer filter pattern is a 2×2 pattern having 1 blue (B), 1 red (R), and 2 green (G) squares. An advantage of using a Bayer filter pattern is that only one sensor is required and all color information can be recorded simultaneously, providing for a smaller and cheaper design. In one embodiment, demosaicing algorithms can be used to convert the mosaic of separate colors into an equally sized mosaic of true colors. Each color pixel can be used more than once, and the true color of a single pixel can be determined by averaging the values from the closest surrounding pixels.

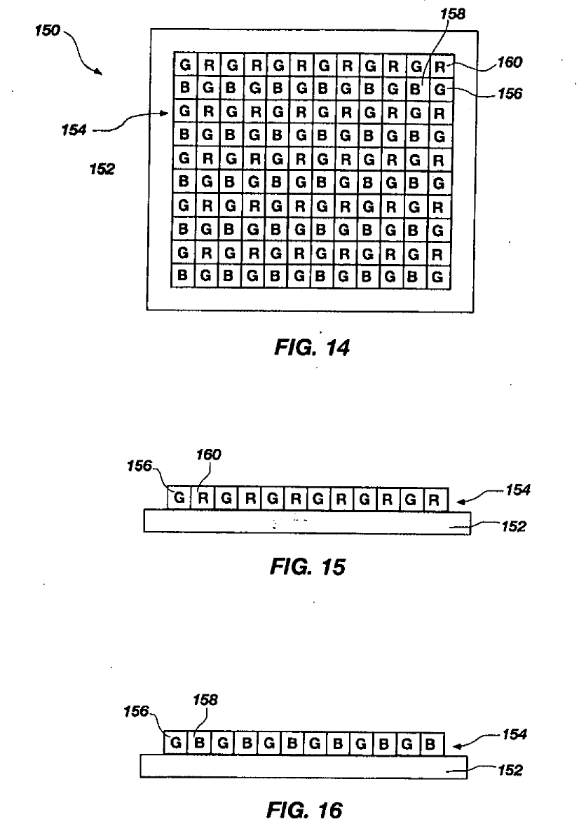

Specifically, with reference to FIG. 14-16, a color filter insert, shown generally at 150, can comprise a substantially optically clear filter substrate 152 and a color filter mosaic portion 154. The filter insert as a whole is made up of green transparent color material 156, blue transparent color material 158, and red transparent color material 160. Each of the transparent color material 156, 158, 160 can be polymerized color resins such as those available from Brewer Science. In one embodiment, the green color material 156 can be put down on the clear filter substrate first, and then the red 160 and blue 158 color material can be positioned in the appropriate spaces provided by the green material. Each transparent color material can be configured to be the size of an SSID image array pixel. The optically clear filter substrate can be, for example, a polymeric material such as SU-8 available from Microchem, having a thickness of about 20 microns, though other thicknesses and materials can be used.

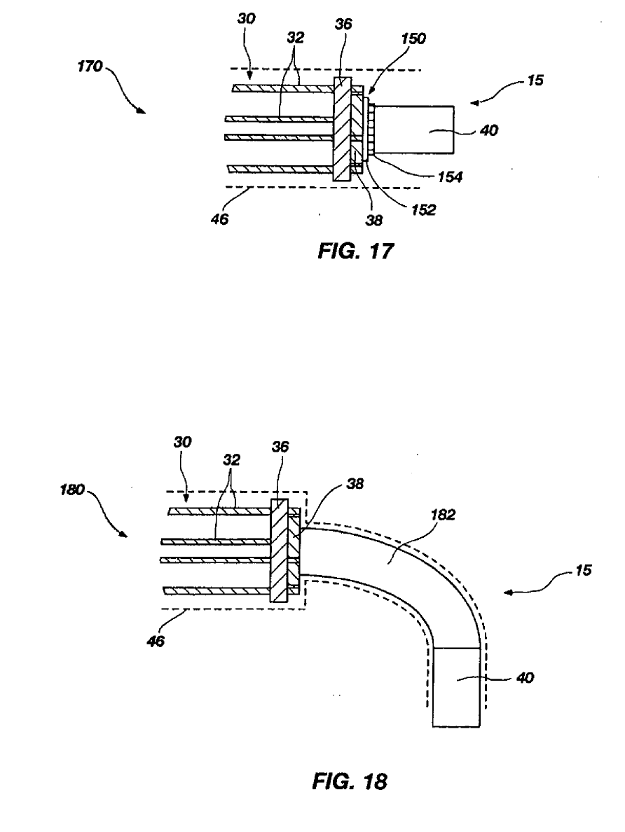

Turning now to FIG. 17, a system 170, including a color filter insert 150 having an optical clear filter substrate 152 and the color filter mosaic portion 154, can be positioned between a GRIN rod lens 40 and an imaging array (not shown) of an SSID 38. FIG. 18 depicts an alternative system 180, wherein a fiber optic 182 is used to optically couple a GRIN rod lens 40 with an imaging array (not shown) of an SSID 38. Any bonding technique or mechanical coupling can be used to connect the SSID to the GRIN rod lens through the color filter insert or fiber optic in order to make the optical connection, such as bonding by an optically clear bonding epoxy. In both FIGS. 17 and 18, as described previously, the imaging device at the distal tip 15 can include a utility guide 36 for supporting or carrying the umbilical 30, which can include electrical wires 32 and other utilities (not shown). Both FIGS. 17 and 18 also depict micromachined tubing 46 to support and direct the camera.

As will be appreciated, an imaging device in accordance with principles of the invention can be made very small, and is useful in solving certain imaging problems, particularly, that of imaging a remote location within or beyond a small opening, for example in human anatomy distal of a small orifice or luminal space (anatomical or artificial, such as a trocar lumen), or via a small incision, etc. In fact, because of the solid state nature of the SSID, and because of the use of the GRIN lens, these cameras can be made to be micron-sized for reaching areas previously inaccessible, such as dental/orthodontics, fallopian tubes, heart, lungs, vestibular region of ear, and the like. Larger lumens or cavities can be view with a greater degree of comfort and less patient duress, including the colon, stomach, esophagus, or any other similar anatomical structures. Additionally, such devices can be used for in situ tissue analysis.

In accordance with an additional embodiment of the present invention, at least one of the micro-cameras comprising the plurality of micro-cameras can comprise a GRIN lens microscope assembly. In this manner, a first imaging system (e.g., GRIN lens micro-camera assembly) may be utilized to observe a wider field of view of a subject and a second imaging system (e.g., GRIN lens microscope assembly) may be used to magnify and carefully examine an area of interest. The first and second imaging systems may be oriented parallel to one another or in a non-parallel fashion but having overlapping field of views. That is, the first and second imaging systems need not be parallel to one another so long as the field of view of the microscope assembly is within the field of view of the micro-camera assembly. In one aspect of the invention, multiple microscope assemblies are disposed within a single field of view of the micro-camera assembly.

In another aspect of the invention, both the first and second imaging systems have adjustable fields of view with respect to the distal end of the catheter. That is, the imaging system itself is movable with respect to the distal end of the catheter. Additional principles of operation and details of construction of similar GRIN lens microscope assemblies can be found in U.S. patent application Ser. No. 12/008,486 filed Jan. 1, 2008 and entitled “Grin Lens Microscope System” which is incorporated herein by reference in its entirety.

An image, or image point or region, is in focus if light from object points is converged almost as much as possible in the image, and out of focus if light is not well converged. For a lens, or a spherical or parabolic mirror, the focal point is a point onto which collimated light parallel to the axis is focused. Since light can pass through a lens in either direction, a lens has two focal points-one on each side. The distance from the lens or mirror's principal plane to the focus is called the focal length. In traditional lens systems, as the length from the distal end of a lens system to a target changes, the distance between moveable lens members is adjusted in order to keep the target “in focus.” That is, the lens members are adjusted to adjust the focal length of the lens system. This is particularly difficult to accomplish when operating miniaturized devices.

In accordance with one embodiment of the present invention, a method of imaging a target using a miniaturized imaging device is disclosed. The method operates based upon the principle that the focal length of a lens is dependent on its refractive index and as such, different wavelengths of light will be focused at different focal lengths. The method comprises providing a miniaturized imaging device (such as those described herein) comprising at least stationary lens system (such as a GRIN lens system) and an imaging array (such as an SSID), wherein the distance from a distal end of the stationary lens system to the imaging array is fixed. The method further comprises advancing the miniaturized imaging device near the desired target and determining a distance from a distal end of the stationary lens system to the desired target. A desired wavelength of light is calculated based on the determined distance from the distal end of the stationary lens system to the desired target and is thereafter propagated onto the target. Thereafter, the desired wavelength of light reflected off of the target is received by the imaging device. In this manner, image focus may be achieved without having to adjust the lens system. Rather, an optimal image focus is determined by an optimal wavelength of light.

In accordance with an additional embodiment of the present invention, a method of imaging a target using a miniaturized imaging device is disclosed comprising, providing a miniaturized imaging device having a stationary lens system and an imaging array, wherein the distance from a distal end of the stationary lens system to the imaging array is fixed. The method further comprises advancing the miniaturized imaging device within a cavity and propagating a starting wavelength of light onto the target within the cavity. The starting wavelength of light reflected from the target is received onto the imaging array. The method further comprises incrementally adjusting the starting wavelength of light to a different wavelength of light and propagating the different wavelength of light onto the target within the cavity. Additionally, the different wavelength of light reflected from the target is received onto the imaging array. An optimal wavelength of light for optimal object focus can be determined using known active and passive autofocus techniques. Other related techniques, structures, and methods of operation are disclosed in U.S. Provisional Application No. 61/084,755 filed Jul. 30, 2008 and entitled “Method and Device for Incremental Wavelength Variation to Analyze Tissue” which is incorporated herein by reference in its entirety.

It is to be understood that the above-referenced arrangements are illustrative of the application for the principles of the present invention. Numerous modifications and alternative arrangements can be devised without departing from the spirit and scope of the present invention while the present invention has been shown in the drawings and described above in connection with the exemplary embodiments(s) of the invention. It will be apparent to those of ordinary skill in the art that numerous modifications can be made without departing from the principles and concepts of the invention as set forth in the claims.

What is claimed is:

1. A miniature imaging device comprising:
a catheter having a distal end and a proximal end;
a first imaging system disposed on the distal end of the catheter, the first imaging system having a level of magnification and a field of view and comprising:
(a) an imaging array disposed on an SSID; and
(b) a GRIN lens disposed on a surface of the imaging array;
a second imaging system disposed on the distal end of the catheter and parallel to the first imaging system, the second imaging system having a level of magnification and field of view and comprising:
(a) an imaging array disposed on an SSID; and
(b) a GRIN lens disposed on a surface of the imaging array; and
wherein the level of magnification of the first imaging system is greater than the level of magnification of the second imaging system and the field of view of the first imaging system is less than the field of view of the second imaging system.

2. The miniature imaging device of claim 1, wherein the imaging arrays of the first and second imaging systems are coplanar.

3. A miniaturized imaging device as in claim 1, wherein the direct contact between the GRIN lens and the imaging array includes a transparent or translucent bonding material at the interface between the GRIN lens and the imaging array.

4. A miniaturized imaging device as in claim 1, further comprising an umbilical, including a conductive line configured for powering and receiving a signal from the SSID.

5. The miniature imaging device of claim 1, wherein the at least two imaging systems provide at least two of: a) increased image resolution, b) increased depth of focus, c) stereoscopic viewing, d) multiple wavelength viewing, e) multiple physical views, and d) image magnification.

6. The miniature imaging device of claim 1, further comprising a plurality of first and second imaging systems disposed circumferentially about a perimeter of the catheter.

7. The miniature imaging device of claim 1, further comprising a plurality of first and second imaging systems disposed longitudinally about an outer surface of the catheter.

8. The miniature imaging device of claim 1, wherein the imaging device is adapted to determining a distance from a distal end of the GRIN lens to a desired target; calculate a desired wavelength of light based on the determined distance from the distal end of the GRIN lens to the desired target, propagate the desired wavelength of light onto the target, and receive the desired wavelength of light reflected off of the target.

9. The miniature imaging device of claim 1, wherein, the imaging device is adapted to propagate a starting wavelength of light onto a target within the cavity, receive the starting wavelength of light reflected from the target onto the imaging array, incrementally adjust the starting wavelength of light to a different wavelength of light, propagate the different wavelength of light onto the target within the cavity, and receive the different wavelength of light reflected from the target onto the imaging array.

10. A miniature imaging device comprising:
a miniature capsule body;
a plurality of imaging arrays disposed on a plurality of SSIDs respectively, the plurality of imaging arrays being positioned about the miniature capsule body to provide a plurality of non-parallel views; and
a plurality of GRIN lenses optically disposed in direct contact with a top surface of the plurality of imaging arrays and configured such that a distal end of each of the GRIN lenses is substantially coplanar with an outer surface of the capsule body.

11. The miniature imaging device of claim 10, further comprising a wireless transmitter adapted to send signals to a remote receiver.

12. The miniature imaging device of claim 11, further comprising a receiver fixedly attached to a location outside the body of a patient, said receiver configured to receive signals from the capsule while said capsule is within the body of the patient.

13. The miniature imaging device of claim 10, further comprising a microscope imaging system disposed on an outer surface of the miniature capsule body, said microscope imaging system oriented to have a focal plane parallel to the focal plane of at least one of the multiple GRIN lenses.

14. The miniature imaging device of claim 10, further comprising a microscope imaging system comprising at least one GRIN lens disposed parallel to at least one of the multiple GRIN lenses disposed on a top surface of the imaging array.

15. The miniature imaging device of claim 10, further comprising a plurality of light sources disposed about an outer surface of the capsule.

16. A miniature imaging device comprising:
a miniature capsule body;
an SSID having a plurality of non-parallel sides, said SSID enclosed within the miniature capsule body;
a plurality of imaging arrays each disposed on a different one of the non-parallel sides of the SSID; and
a plurality of GRIN lens each optically coupled to a different one of the plurality of imaging arrays and oriented substantially within the capsule body.

17. The miniature imaging device of claim 16, wherein a top surface of each of the imaging arrays is equidistant from a center of the SSID.

18. The miniature imaging device of claim 16, wherein each of the GRIN lenses is disposed within apertures disposed about an outer surface of the capsule.

19. The miniature imaging device of claim 16, wherein the miniature capsule body comprises a plurality of transparent sections.

20. The miniature imaging device of claim 19, wherein each of the GRIN lenses is disposed directly behind one of the plurality of transparent sections, respectively.
